(19) United States
(12) Patent Application Publication
(10) Pub. No.: US 2025/0256000 A1
(43) Pub. Date: Aug. 14, 2025

(54) DEVICE AND METHOD FOR QUALITY CONTROL OF HAND HYGIENE AND FOR DISINFECTION OF HANDS

(71) Applicant: SVAI EHF., Kopavogur (IS)

(72) Inventors: Alexander JOHÖNNUSON, Kopavogur (IS); Helga Dagny SIGURJONSDOTTIR, Kopavogur (IS)

(21) Appl. No.: 18/867,348

(22) PCT Filed: May 23, 2023
(86) PCT No.: PCT/IS2023/050004

§ 371 Date: Nov. 19, 2024

(30) Foreign Application Priority Data
May 20, 2022 (IS) 050368

(51) Int. Cl. A61L 2/24; A61L 2/00; A61L 2/10; A61L 2/22

(52) U.S. Cl. CPC A61L 2/24; A61L 2/0088; A61L 2/10; A61L 2/22; A61L 2202/14; A61L 2202/15

(57) ABSTRACT

Disclosed is a hand sanitising and hand hygiene monitoring device that provides automatic sanitation of hands, that is, dispensing and full-coverage distribution of sanitation agent without need for hand rubbing. The device comprises a housing comprising a hand sanitisation and sensing area in which the hands are located for sanitising; a sensing arrangement configured to obtain data relating to a characteristic of the user's hand located within the sanitisation and sensing area; and, a dispensing device for dispensing and distributing a sanitising agent in the sanitising and sensing area and over the hands. The device and system can monitor compliance with sanitation protocols, including monitoring and alerting if non-allowed external items are used (rings, watches) and identify and alert for other compromising details, such as nail polish, wounds, and dirt.

### TECHNICAL FIELD OF THE INVENTION

The present disclosure relates to a hand sanitising and hand hygiene monitoring device for sanitising hands with a sanitising agent and an associated monitoring and quality control system. Specifically, an integrated IoT-enabled system allows monitoring and streamlining hand hygiene practices of healthcare workers and provides fully automatic hand sanitation, without need of rubbing hands.

### BACKGROUND TO THE INVENTION

Healthcare acquired infections, HCAI's, such as Covid-19, urinary tract infections and infections following surgery in healthcare settings have been linked to ineffective hand hygiene of medical personnel and caretakers resulting in direct contact transmission of pathogens methods (the most common transmission route for HCAI pathogens).

As such, the use of hand sanitising and hand hygiene monitoring devices which deposit an amount of hand sanitising agent on a user's hands have become well known in healthcare facilities (and elsewhere). Attempts have been proposed in the art for automatically dispensing sanitation liquid to hands. Some devices use image sensors for detecting hands and eventual foreign objects and some use sensor feedback to adjust dispensed amount of sanitation agent, sec e.g. CN 107874676 A and CN 108732975 A. Neither of these prior art documents, however, ensure automatic sanitation, as the user himself must thoroughly distribute the sanitation agent on his or her hands, and this is a critical component of the sanitation process.

Conventional hand-rubbing protocols require that the person follows a thorough set of instructions, typically the instructions include rubbing hands palm to palm; with fingers interlaced, rubbing palm to palm and then right palm to back of left hand and vice versa; cusping back of fingers into opposing palm and rub side to side; clasping right hand around left thumb and rub thumb in rotational manner and vice versa; rubbing in a rotational manner backwards and forwards by placing fingertips of right hand in left palm and vice versa. Thus, thorough and correct rubbing takes time, according to World Health Organisation's recommendations and instruction posters 20-30 sec. This must be repeated every time a healthcare worker has exposed his hands to potential contamination and before a next task that requires clean hands, e.g. when going from one patient to another, if hands need to exposed and used. Studies show that it remains a challenge to ensure that proper rubbing technique is used every time repeated sanitation is needed.

The present invention seeks to provide an improved hand (and wrist) sanitising device and associated system that allow the devices to be used also for hand hygiene monitoring and quality control.

### SUMMARY OF THE INVENTION

The present invention provides a hand sanitising and hand hygiene monitoring device and a hand sanitising system according to the appended claims.

The device of the invention provides fully automatic dispensing and proper and complete distribution of sanitation agent to hands so that the hands and wrists become sanitised, and the user does not need to manually distribute the agent by rubbing. The device thus prevents uneven and incomplete distribution that could compromise sanitation. By the term “complete distribution” is meant full and effective surface coverage so that hands are fully and properly sanitised.

The device and system can further monitor compliance with sanitation protocols, including monitoring and alerting if non-allowed external items are used (rings, watches) and identify and alert for other compromising details, such as but not limited to nail polish, artificial nails (gel nails and acrylic nails), wounds, dirt, etc.

In a first aspect, the present disclosure provides a hand sanitising and hand hygiene monitoring device that automatically sanitises hands, comprising a hand sanitisation and sensing area in which the hands and preferably also the wrists are located for sanitising and for sensing. The hand sanitisation and sensing area may be provided within a housing of the hand sanitising and hand hygiene monitoring device.

The hand sanitising and hand hygiene monitoring device may further comprise a dispensing device configured to dispense an amount of sanitising agent in the sanitisation and sensing area and distribute on the user's hand. (Whenever sanitation of the hand or “hand sanitising and hand hygiene monitoring device”, according to the present invention is mentioned herein, this preferably also includes sanitation of the wrist and a device that preferably sanitises the hand and wrist.) The dispensing device comprises at least one nozzle for dispensing hand sanitising agent onto the hand.

The hand sanitising and hand hygiene monitoring device comprises a sensing arrangement configured to obtain data relating to a characteristic of the user's hand located within the sanitisation and sensing area. The characteristic may be any characteristic relating to the user's hand. The characteristic may be useful for aiding the dispensing of a sanitising agent and/or for monitoring the depositing of a sanitising agent and/or for monitoring the use of the hand sanitising and hand hygiene monitoring device and/or for determining a hygiene level of a user's hand. The characteristic may relate to but is not limited to one or more from the group comprising: a position of the user's hand relative to the dispensing device; a size of the user's hand; a contaminant on the user's hand; a wound on the user's hand; and, a foreign object on the user's hand. The position of a user's hand may comprise the position and orientation relative to the dispensing device and/or the posture of the hand.

The contaminant may comprise one or more taken from the non-exhaustive group comprising: faecal matter; urine; blood; other bodily fluids; dirt; and nail polish. The foreign objects may comprise one more taken from the non-exhaustive group comprising jewellery and clothing (including woven wrist bands and the like). The jewellery may comprise rings, bracelets or watches, for example. The wounds may comprise one or more lacerations or abrasions.

The hand sanitising and hand hygiene monitoring device may further comprise: a user interface. The user interface may be configured to provide information relating to the above-mentioned characteristic to the user. The user interface may comprise a device for providing an audible output and/or a visual output. The visual output device may comprise a display device comprising a screen configured to provide one or more images of a user's hands. The audio output device may comprise a speaker, for example.

The user interface may further include a device for receiving a user input, such as but not limited to a keyboard, a touchscreen, a scanner or sensor for scanning/sensing a user ID or other input data from a user tag (such as barcode, QR code, an RFID tag, smart tag or the like), or a microphone for receiving audible user input (spoken instructions or responses).

The user interface may be configured to provide positional information to the user. For example, the display screen may be configured to provide an image of the hand in relation to a preferred position for scanning and/or dispensing. The user interface may optionally provide directions for a user to reposition their hand. The directions may be visual including one more prompts, such as arrows, or alignment marks against which the user can readjust the position of their hands. The directions may be audible and instruct the user to reposition their hands in a particular way, for example, “move hands up” or “insert hands further into the sanitisation and sensing area”.

The user interface may be configured to provide instructions to a user to remove one or more foreign objects. For example, where the hand sanitising and hand hygiene monitoring device determines the presence of one or more foreign objections which may interfere with the sanitisation process, the hand sanitising and hand hygiene monitoring device may instruct a user to remove the foreign object.

The user interface may be configured to provide information or instructions in relation to sensed contaminants (determined suspected contaminants based on the obtained image data) or foreign objects. For example, the user interface may be configured to provide a user with an image or representation of their hand indicating the location of suspected contaminants or foreign objects.

The user interface may also or alternatively be configured to provide statistical information to the user and/or to a central server as described below, such as how often he or she has used the device or system that day, how his/her cleanliness has met the required criteria, etc.

The user interface may comprise a display device provided on the housing or adjacent the housing. The display device may be provided on an upper surface of the housing and may be provided on a front surface of the housing.

The housing may comprise an opening into which the user can insert their hand into the sanitisation and sensing area. The opening may be located on a front or upper side of the housing. Advantageously, the form and configuration of the opening(s) is arranged so as to optimise the performance of the device while accommodating ergonomic aspects for the user. The sanitisation and sensing area may be enclosed by walls of the housing on two or more sides, or three or more sides, or four or more sides or five or more sides. The hand sanitising and hand hygiene monitoring device may be configured to receive a user's hand horizontally or vertically or at an angle between horizontal and vertical. The sanitisation and sensing area may be suitable for receiving one or both of a user's hand. The hand sanitising and hand hygiene monitoring device may comprise two sanitisation and sensing areas, one for each hand, or a single hand sanitisation and sensing area which is configured to receive both hands simultaneously.

In order to make the device more slim, the housing can in some embodiments comprise a wall unit configured to be fastened on a wall or stand, and a moveable (foldable) frame that is joined to the wall unit on hinges or the like fastening arrangement such that the frame can fold out from a substantially vertical position (“closed” position) to an open position (“active” position), in which the user can put his hands through the frame. The frame can thus be seen as comprising walls, on at least three sides that define a sanitation space (sanitisation and sensing area). The frame comprising the walls typically comprises one or more of the at least one sensor (e.g. camera sensor(s)). In this embodiment, the user may be instructed to slowly move his hands through the frame during the sanitation and sensing, in order to ensure full and complete surface coverage. Additionally, the device can be configured to move the frame during the sanitation cycle, e.g. from a downwardly pointed angled orientation to an upwardly pointing angled orientation or vice versa, as part of the sanitation method, to ensure full hand surface coverage.

The sensing arrangement may comprise a plurality of cameras configured to capture images of the user's hand or hands when in the sanitisation and sensing area. The plurality of cameras may be configured to obtain a plurality of different images from different angles.

The cameras may be configured to image the palmer side and/or the dorsal side of the user's hand. There may be a plurality of cameras located on a side of the sanitisation and sensing area which corresponds to the palmer side and/or dorsal side of the hand. The cameras may be located on an upper surface and/or a lower surface of the sanitisation and sensing area. There may be a plurality of cameras for imaging each hand. The cameras may be configured to obtain image data which may be used to construct a three-dimensional image of the user's hand. From such 3D representation can be derived full surface area of the hands (size of the surface area), which in turn can be helpful for proper sanitation (e.g. as input for controlling/adjusting suitable dose).

The plurality of cameras may comprise at least one camera configured to detect light having a wavelength of between 10 nm and 400 nm. The camera may comprise one or more filters configured to remove one or more wavelengths of light. The device may further comprise one or more light sources for illuminating the sanitisation and sensing area with a light having a predetermined wavelength.

The cameras may comprise one or more lens or lens cover through which an image is captured. The lens or lens cover may be configured to prevent the accumulation of hand sanitising agent thereon. Such protective lens maybe an additional lens to the main camera lens (focusing lens). The mentioned lens may comprise one or more hydrophobic coatings. The lens may comprise a heater configured to evaporate hand sanitising agent from the lens. The lens may comprise a mechanical cover which is operable to cover the lens during dispensing of the sanitising agent. The lens may comprise one or more wipers configured to remove sanitising agent from the lens. The wiper may be operated on a periodic basis to remove the sanitising agent.

The hand sanitising and hand hygiene monitoring device may further comprise a UV light source for sterilising the enclosure (i.e. interior walls of the hand sanitisation and sensing area). The UV light may be shielded by a shield arranged between the opening of the sanitisation and sensing area and the UV light. The UV light may be operable at predetermined times when the device is not being used (for example, outside of normal working hours or as part of a maintenance schedule).

The device may further comprise at least one processor configured to determine the characteristic from the obtained data. The at least one processor may be configured to construct an image of the user's hand including indicia relating to the characteristic. The images may comprise indicia relating to hand position, foreign objects, contaminants or a wound, for example. The images may comprise a representation of the hand showing areas where contaminants or foreign objects have been detected. The images may be three-dimensional images of the hand.

The device may further comprise at least one memory. The memory may store instructions which, when executed by the processor cause the performance of any of the method steps disclosed herein.

The dispensing device may further comprise at least one nozzle for dispensing sanitising agent onto the hand. The at least one nozzle may comprise a spray head for ejecting a spray of sanitising agent. The nozzle may comprise an air assisted atomising nozzle or an ultrasonic nozzle, for example. The nozzle may be a directional nozzle so to eject hand sanitising agent in predetermined direction. The directional nozzle may be variably adjusted to allow the predetermined direction to be changed prior to or during dispensing of the sterilising agent. The predetermined direction may be determined and automatically adjusted using the obtained data or characteristic. The at least one nozzle preferably comprises at least one nozzle directed to each side of an in inserted hand, i.e. at least one nozzle directed to the palmer side and at least one nozzle directed to the dorsal side, e.g. two or more nozzles directed at each side. The nozzles may be arranged so that they are moveable, such as on a slidable member. Thus, in some embodiments the at least one nozzle can be moved based on obtained data defining the position and/or size of the hands. In one embodiment, one or more nozzle are arranged on at least one diagonal member that is slidable along rails. In some embodiments one or more nozzle are arranged on a substantially circular or elliptical path, such that one or more nozzle ca be moved in a path around one or both hands.

The hand sanitising and hand hygiene monitoring device may further comprise a controller configured to control the amount and/or a spray pattern of the sanitising agent. The amount and/or spray pattern may be determined using the obtained data or characteristic. Thus, the device and methods described herein can thereby ensure full surface coverage of the fluid media onto the user's hands and wrists, ensuring that no areas are left compromised.

The hand sanitisation device may comprise a user identifier device. The user identifier may comprise a camera configured to capture an image of a user's face, or an electronic reader configured to read a tag provided by a user, such as mentioned above. The hand sanitisation device may be configured to determine a user identity using an image of the user's face using a facial recognition algorithm or an image or images of a user's hand or hands, using a suitable recognition algorithm e.g. fingerprint recognition, and/or data read by the reader.

The hand sanitising and hand hygiene monitoring device may be remotely connected to a server. The server may be a remote computing device or computer program provided on another computing device. Thus, in some embodiments the server may refer to “cloud-based” software functions. The server may be referred to as a central hub. The server may be configured to receive the obtained data from one or more devices of the invention. The server may be configured to provide information in relation to the obtained data and/or characteristic of hand.

The server may be configured to carry out any of the data processing steps disclosed herein. Hence, the device may be configured to obtain the data relating to the characteristic or user identification and provide the obtained data to the server for processing. Hence, the device may further comprise a transmitter or wired connection configured to transmit the obtained data or data relating to the characteristic to the remote server. The server may be configured to receive the obtained data and determine the characteristic and/or user identity.

The device may be configured to receive data from the server. The received data may relate to the characteristic and/or user identity. The received data may comprise audio data or image data for displaying to a user. The image data may comprise an image of the user's hands. The image data may comprise a three-dimensional image or representation of the user's hand which may optionally include indicia of foreign objects or contaminants or wounds.

The device and/or server may be configured to process the obtained data using a machine learning algorithm. The machine learning algorithm may be configured to determine the hand position or the characteristic.

In some embodiments the server is connected to a plurality of devices such as for an entire hospital. Thus, the invention can provide a hand sanitation Quality Monitor System (QMS), which is preferably a cloud-based QMS. It is advantageous to have such system incorporating machine learning features (artificial intelligence; AI) as the AI can tune the system for particular premises, For example, when the system identifies each user (as discussed above), it can create personalised sanitation profiles for each healthcare worker, considering individual variables, such as hand size, commonly worn jewellery, and/or typical hand dryness level of the respective individual. Over time, an Al system will tune and adjust these profiles, to ensure optimal amount, dispensing time, distribution pattern etc. for each recognised user.

The devices connected to a cloud-based server may referred to as IoT (internet of things) devices and the system as an IoT system. IoT typically refers to physical objects (or groups of such objects) with sensors, processing ability, software and other technologies, that connect and exchange data with other devices and systems over the Internet or other communications network.

Thus, innovative approaches, powered by AI, aim to enhance the efficacy, convenience, and personalization of the hand sanitization process of the invention. The possible additions can be incorporated seamlessly, offering an advanced, comprehensive solution for hand hygiene management.

In another aspect, the present disclosure provides a hand sanitising system comprising any hand sanitising and hand hygiene monitoring device disclosed herein and any server as disclosed herein.

The server may be configured to provide information to an end user in relation to the obtained data and/or characteristics of one or more users. The information may comprise statistical information. The information may comprise one or more taken from the group comprising: a number of users; a number of times of use; a time(s) of a use; locations of use (where there are multiple devices within a location or premises); the type and/or amount of contaminants for each user; the type and/or size and/or location of wounds; and, the type and/or location and/or size of foreign objects on the user's hand.

The system may further comprise one or more further computing devices from which the information may be viewed by an end user. The end user may be a manager, agent or owner of a facility in which the device is installed. The information may be used to determine the efficacy of hand sanitisation within a healthcare facility and/or provide causative links between hand hygiene and/or hand sanitisation in the facility and healthcare acquired infections.

In a further aspect, the present disclosure may provide a server configured to receive obtained data from a hand sanitising and hand hygiene monitoring device of the invention. The obtained data may comprise one or more images of a user's hand located within a sanitisation and sensing area of the device. The one or more images may comprise different images having different wavelengths. The server may be configured to determine one or more characteristics of the user's hand using the obtained data. Determining the one or more characteristics may comprise the use of a machine learning algorithm. The server may be configured to transmit the characteristic to the device from which it received the data.

The obtained data may comprise user identification data. The user identification data may comprise an image of a user's face or an identifier such as a name or number. The server may be configured to determine a user's identification using the identification data. The server may be configured to execute a facial recognition algorithm on the image of the user's face.

In a further aspect the present disclosure provides a method of sanitising a user's hand. The method may comprise the use of a hand sanitising and hand hygiene monitoring device disclosed herein. The method may comprise: the device obtaining data from a user's hand placed within a sanitisation and sensing area of the device; and dispensing an amount of sanitising agent on the user's hand. The obtained data may comprise one or more images of the user's hand and/or one or more user identifier such as those described above.

The dispensing of the sanitising agent may be determined from the obtained data. The method may further comprise processing the obtained data to determine one or more characteristics of the user's hand. The characteristic may be any characteristic as disclosed herein. Determining the characteristic may comprise the use of a machine learning algorithm. The determination of the characteristic may be carried out by a server which is remote to the device.

In a further aspect the present disclosure provides a method of sanitising a user's hand. The method may comprise the use of a hand sanitising and hand hygiene monitoring device disclosed herein. The method may comprise: the user inserting a hand into the sanitisation and sensing area of the device; the user engaging with a user interface of the device and responding to instructions thereon; the user receiving an amount of hand sanitising agent dispensed from a dispensing device of the device.

The instructions may relate to the repositioning of the hands or foreign objects which require removal. The instructions may comprise one or more images of the hands showing the position of the hands relative to a desired position, or contaminants or foreign objects on the hands.

The skilled person will appreciate that except where mutually exclusive, a feature described in relation to any one of the aspects, embodiments or examples described herein may be applied mutatis mutandis to any other aspect, embodiment or example. Furthermore, except where mutually exclusive, any feature described herein may be applied to any aspect and/or combined with any other feature described herein.

### BRIEF DESCRIPTION OF THE DRAWINGS

In order that the invention may be more clearly understood one or more embodiments thereof will now be described, by way of example only, with reference to the accompanying drawings, of which:

FIG. 1 shows a perspective view of a hand sanitising and hand hygiene monitoring device;

FIG. 2 shows an alternative view of the device of FIG. 1;

FIG. 3 shows the device of FIGS. 1 and 2 with a user's hands located in a sanitisation and sensing area;

FIG. 4 shows a cross section of the device of FIGS. 1 to 3;

FIG. 5 shows an alternative hand sanitising and hand hygiene monitoring device with the hand sanitising and sensing area open upwardly and sideways;

FIG. 6 shows a different view of the device from FIG. 5;

FIG. 7 shows the device from FIG. 5 with a user's hands located in a sanitisation and sensing area;

FIG. 8 shows a top view of the device from FIG. 5, with inserted hands;

FIG. 9 shows the device from FIG. 5 with part of the front panel of the housing removed;

FIG. 10 shows the device of FIG. 5, with a front portion of the housing removed;

FIG. 11 shows another alternative hand sanitising and hand hygiene monitoring device;

FIG. 12 shows a system comprising a plurality of devices and a server; and,

FIG. 13 shows a schematic of a device.

FIG. 14 shows another embodiment of the device, with a foldable frame that defines the sensing and sterilisation area.

FIG. 15 shows a top-view of the embodiment from FIG. 14.

FIG. 16 shows the same embodiment as FIGS. 14 and 15, where the frame has been folded to a closed position.

### DETAILED DESCRIPTION OF THE INVENTION

In the following detailed description, numerous specific details are set forth in order to provide a thorough understanding of various embodiments and the inventive concept. However, those skilled in the art will understand that: the present invention may be practiced without these specific details or with known equivalents of these specific details; that the present invention is not limited to the described embodiments; and, that the present invention may be practiced in a variety of alternative embodiments. It will also be appreciated that well known methods, procedures, components, and systems may have not been described in detail.

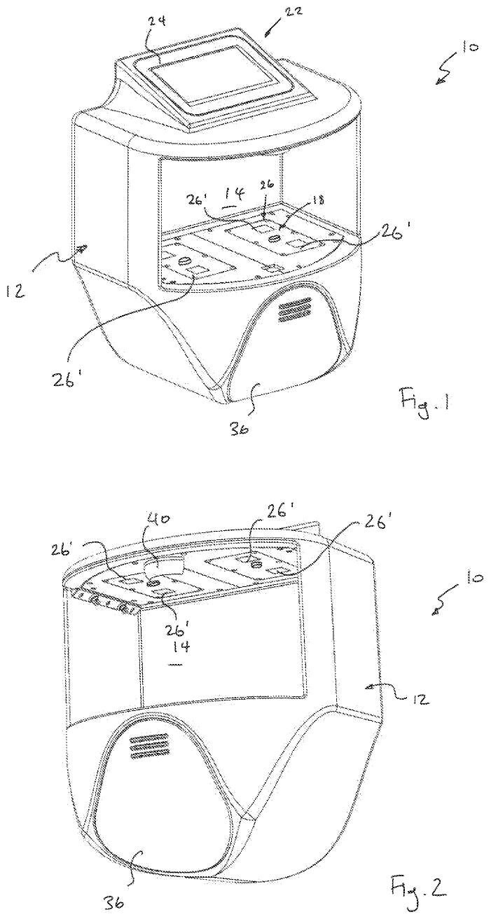

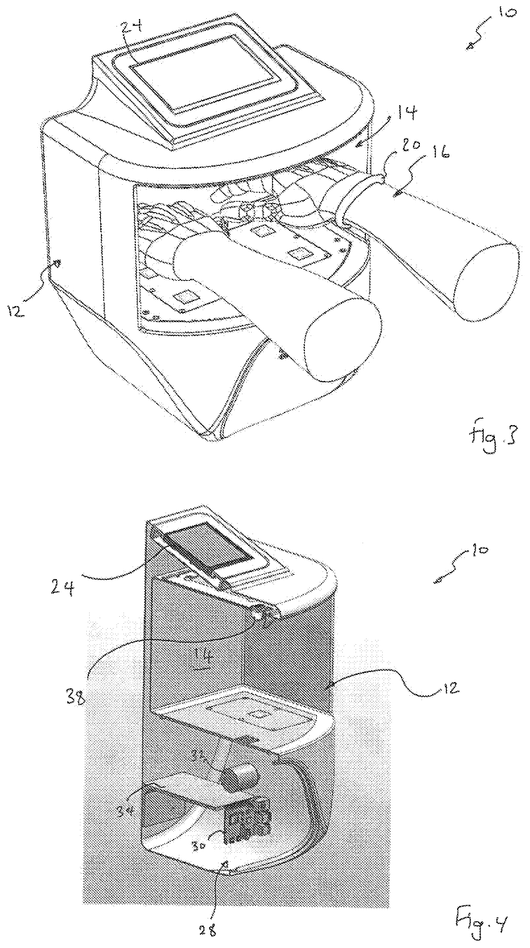

FIGS. 1 to 3 show various views of a hand sanitising and hand hygiene monitoring device 10 according to the present disclosure. FIG. 4 shows a sectional view of the device.

The hand sanitising and hand hygiene monitoring device 10 may generally comprise: a housing 12 comprising a hand sanitising area 14 for the locating the hands 16 of a user for sanitising; a sensing arrangement configured to obtain data relating to a characteristic of the user's hand located within the sanitisation and sensing area; and, a dispensing device 18 for dispensing a sanitising agent in the hand sanitising area and onto the hands 16.

### General Statements

The present disclosure seeks to provide a hand sanitising and hand hygiene monitoring device 10 which can improve the efficacy of sterilizing in healthcare settings (or elsewhere). This may be achieved by, for example, providing instructions to users either generally or in response to sensed characteristics of their hands, collecting data in relation to users who are sterilising their hands, and/or improving the dispensing of a sanitising agent to ensure it is more effective and efficient.

The device 10 of the present disclosure may be configured to obtain data relating to one or more characteristics from a user's hands 16. For example, in some embodiments, the device 10 may be configured to obtain data relating to the position of the user's hands 16. Hence, if the hands 16 are not positioned correctly in relation to the dispensing device 18, they may be repositioned prior to sanitising agent being dispensed. It will be appreciated that the term “user's hand” within this disclosure may relate to one or both hands and may include the hands and wrist.

In some embodiments, the hand sanitising and hand hygiene monitoring device 10 may be configured to obtain data relating to surface contaminants on the hands 16. Contaminants, such as urine, faecal matter, blood, nail polish and/or dirt may reduce the effectiveness of the sanitisation process, either by preventing the sanitising agent contacting the skin or by harbouring excess pathogens—a biofilm, which can prevent or reduce effective sanitation. Being able to detect such contaminants can be useful for understanding HCAIs in a particular healthcare setting and/or for directing users to improve their hand cleansing routine and/or for allowing the sanitising agent to be applied in a more appropriate way.

In some embodiments, the device 10 may be configured to detect foreign objects on the hands 16 such as jewellery or items of clothing, e.g. rings, watches, bracelets and sleeves, which may obscure parts of the hands 16 or make sanitising generally more ineffective. The acquired data may be used to obtain a better understanding of HCAIs and/or for directing users during a sterilisation procedure, e.g. to remove jewellery, and/or to allow for a sterilising agent to be applied in a more appropriate way.

In some embodiments, the device 10 may be configured to detect one or more wounds, such as lacerations, abrasions or other skin conditions which comprise a breakdown in the integrity of the skin, e.g. rashes. Wounds may generally comprise areas where contaminants and/or pathogens can accumulate and so understanding where and when they are present in users can provide an important tool for improving understanding of HCAIs.

In short, providing a device 10 which is configured to acquire data relating to one or more characteristics of a user's hands 16 can be beneficial for understanding and improving hand sanitisation and also the role hand sanitisation may have in a particular healthcare setting in terms of addressing and preventing HCIAs. As noted above, particularly useful data may relate to positional data of the hands, surface contaminants, the presence and position of foreign objects and the presence and position of wounds, but others may exist and this list should not be taken as being exhaustive.

In some embodiments, the obtained data may be used to provide information to a user to improve a sterilisation process. For example, where the hands 16 are located in the wrong position, either in relation to the position within the sterilisation area, i.e. the depth of the insertion, lateral positioning, height, angle (tilt or yaw), or the posture, e.g. the flexion or extension and/or spread of the fingers, then a user may be directed to reposition the hands 16. In embodiments where surface contaminants or foreign objects are detected, then a user may be instructed to take corrective action prior to sterilisation. For example, a user may be requested to remove jewellery or to wash and/or dry their hands before proceeding.

In some embodiments, the data may be used to alter how the sanitising agent is applied. For example, the obtained data may comprise size data in relation to the hands, or the number, type, amount or location of contaminants or foreign objects which may determine the amount of sanitising agent which should be deposited.

In embodiments where the dispensing device 18 comprises a plurality of nozzles and/or directional nozzles for dispensing sanitising agent, the obtained data may be used to determine how the nozzles are controlled to dispense the sanitising agent in a predetermined pattern or volume, possibly in specific locations. Hence, any area which includes a foreign object or higher levels of contaminants may receive a higher dose of sanitising agent. In some embodiments, knowledge of the user's hands may allow the sanitiser to be applied more sparingly to help reduce any associated skin irritation and/or waste.

In some embodiments, the hand sanitising and hand hygiene monitoring device may be configured to determine whether the application of the hand sanitising agent is satisfactory. This may be achieved by imaging the deposition of the hand sanitising agent on the hands, for example, where the sanitising agent is deposited as a spray, or may be achieved by analysing the position of the hands relative to the dispensing nozzle or nozzles. The dispensing nozzle may comprise an air assisted atomising nozzle or an ultrasonic nozzle, for example.

The sanitising agent may be any suitable sanitising agent known in the art. The sanitising agent is typically selected and/or formulated to have a suitable viscosity to attain effective and efficient distribution of the agent with the selected dispensers (nozzles or the like). Thus the sanitising agent may, for example, be ethanol or another skin-tolerated sanitising agent or combination thereof, suitably formulated in particular to obtain desired viscosity.

In some embodiments, the device 10 may be provided with a user interface 22. The user interface 22 may include visual and/or audible outputs for conveying information to a user. The visual output may be in the form or a display device 24, such as an LCD or LED screen, configured to show images or moving pictures. For example, the user interface 22 may provide directions for repositioning the user's hands 16 with images and/or words.

The display device 24 may be configured to display images of the user's hands with contaminants or foreign objects highlighted that require removal. In some embodiments, the user interface 22 may also be used to provide advertising or public health information when the device 10 is not in use. The user interface 22 may comprise an audio output device such as one or more speakers (not shown). The speakers may be configured to provide an audible output for providing instructions or information similar to that described herein for the display device.

In some embodiments, the data obtained by the sensing arrangement may be used to determine statistical information relating to the use and/or users of the device 10 or a plurality of devices. The statistical information may be useful for determining or better understanding HCAI's in a particular setting or for a group of users. The data may be used by the owner, agent or manager of a facility where the device 10 is installed so as to understand the behaviours of the user population. For example, data relating to the number of users, times of use, locations of use (where there are multiple devices within a location or premises), levels of contaminants, wounds or foreign objects. This data may be used to understand and improve a healthcare facility's hand sanitising practices and/or to identify causative links for HCAIs, to better minimize or prevent HCAIs. It will be appreciated that any user data gathered may be made anonymous in accordance with local data protection regulations and/or local policy.

In some embodiments, the hand sanitising and hand hygiene monitoring device 10 may comprise one or more imaging system 26 configured to image the user's hands 16. The images of the user's hands 16 could be used to determine one or more characteristics of the user's hands. The imaging system may be configured to detect visible light only, or may be configured to detect multi-spectral images comprising a range or number of ranges of wavelengths. Multi-spectral images may be beneficial for the identification of the hand position and/or contaminants and/or foreign objects. The multispectral imaging may include, for example, wavelengths of between 10-400 nm light.

The hand sanitising and hand hygiene monitoring device 10 may comprise light sources for producing the required light required by the imaging system.

The device 10 may comprise one or more processors or controllers. The one or more processors or controllers may be configured to receive data from the sensing arrangement and determine, using the data, one or more characteristics of the user's hands 16. The characteristics may comprise one or more from the group comprising: hand position and/or posture; the presence and/or location of contaminants; the presence and/or location of foreign objects; and, the presence and/or location of a wound on the user's hands.

In some embodiments, the device 10 may be configured to control the dispensing of sanitising agent via the dispensing device 18. The controller may be configured to determine a location of dispensing and/or an amount of sanitising agent to dispense.

The one or more controllers may execute a machine learning algorithm. The machine

learning algorithm may be used in the detection of hand position and/or foreign objects and/or contaminants, for example. The machine learning algorithm may be employed within the device 10 so as to provide more efficacious application of the device and to determine the distribution of the sanitiser, for example, in relation of a hand position and posture, and/or contaminants, and/or foreign objects, and/or wounds.

In some embodiments, the device may be configured to determine when a sanitising procedure is complete. Confirmation of the completed sanitising process may be communicated to the user via the user interface. In some embodiments, the duration of the hand sanitising process may be less than 10 seconds, such as within a range of 5-10 seconds or even less than 5 seconds from the user inserting their hands into the sanitisation and sensing area to the dispensing of the sanitising agent or confirmation that the sanitising process is complete.

In some embodiments, the device 10 may be communicatively connected to a server (which may be referred to as a hub) which can be a local physical server (where “local” can mean that the server is in the same general facility, such as e.g. in the same hospital facility, as the device, and not necessarily directly adjacent or in the same room) but can as well be cloud-based or any other type of provided server-service. The server may be used to receive data from one or more devices and use the data to determine one or more characteristics of the user's hands. The characteristics and/or commands relating to the characteristics may be provided to the device for use in the sanitisation process. For example, the data received from the hub may inform how the sanitiser agent is dispensed and/or instructions for the user to follow prior to or during the sanitisation process.

The server may be configured to determine statistical information for the use of the or a plurality of devices. The statistical information may be any information useful for analysing the use and/or efficacy in a healthcare setting (or elsewhere).

In some embodiments, the data obtained by the hand sanitising units may be used to train a machine learning algorithm.

### Sanitisation and Sensing Area—Specific Detail

Referring to the hand sanitising and hand hygiene monitoring device shown in FIGS. 1 to 3 in more detail, the sanitisation and sensing area 14 may be provided as a cavity within the housing 12 into which a user can readily insert their hands 16. In the embodiment shown, the cavity is enclosed on five sides by the walls of the housing 12 with a sixth side being fully open and facing the front of the sanitising device 10 in the direction from which a user would ordinarily approach. The cavity may be located centrally within the housing 10 and may comprise the sensing arrangement and dispensing device mounted to a wall thereof. Hence, for example, the sanitising area 14 may comprise the parts of the sensing arrangement and/or dispensing device 18 on upper and/or lower walls thereof. As such, the hands 16 may be sensed, e.g. imaged, and/or receive sanitising agent from above and/or below, as required.

The device 10 shown in FIGS. 1 to 3 is a wall mounted unit, however, this is not a limitation and the unit may be mounted on a free-standing pedestal or elsewhere, as desired. Thus, the housing 12 may include any mounting features for mounting the device 10 either to a wall or pedestal as may be required or known in the art. For example, a rear panel of the housing 12, may comprise one or more apertures through which a fixing element such as a screw or bolt may be received.

The housing 12 may further comprise at least one internal cavity 28 in which to store one or more of a supply of sanitising agent, a pump for pumping the sanitising agent or other means to deliver sanitising agent from the supply, and any electrical equipment or electronic circuitry required for operating the device. The supply can be in the form of e.g. a canister or cartridge, and the pump function may be provided by an electric pump arranged in the housing and which is connected to the canister, in another embodiment the pump function is integrated in the cartridge, and such integrated pump function could be activated e.g. by a motorized or electro-mechanical component, e.g. a motor or solenoid. Thus, as can be seen in the cross-section of FIG. 4, there is provided a cavity 28 located beneath the sanitisation and sensing area 14 in which there is located a printed circuit board 30 which may include any necessary controllers, processors and memory for the operation of the device 10, a canister 32 comprising the sanitising agent. The cavity 28 may be provided with any required supporting structure 34 or partitions required. The electrical equipment may comprise a power supply unit which is connected to an external power supply in use or a battery power supply, and which is configured to provide power to the electrical and electronic equipment within the device such as the controller and user interface, e.g. display device 24. The electronic devices may be that required for imaging the sanitisation and sensing area, data processing for determining the characteristics of the hands, for controlling the dispensing device and/or for generating data for outputting by the user interface.

As shown in FIGS. 1 to 3, the cavity 28 may be provided with an access panel 36 (which is removed in FIG. 4) which may be attached with suitable fixings. The fixings may be configured to prevent unauthorised access, i.e. by including one or more locks or by requiring specialist tools for removal.

In the embodiments of FIGS. 1 to 4, the user interface 22 comprises a display device

24 which is integrated into an upper surface of the housing 12. The display device 24 is received within a suitable aperture provided in the housing and is angled upwards and forwards so as to be presented more favourably to a user standing in front of the device. A cavity may be provided below the display device for location of the dispensing device or sensing arrangement and any cabling or pipework.

In some embodiments the device 10 may comprise a sterilising light source 38 for irradiating the sterilisation area which may be activated at prescheduled times. The sterilising light source 38 of the embodiment of FIGS. 1 to 4 is provided on an upper wall of the sanitisation and sensing area 14 behind a shield 40 which prevents light being emitted directly out of the housing into the surrounding area.

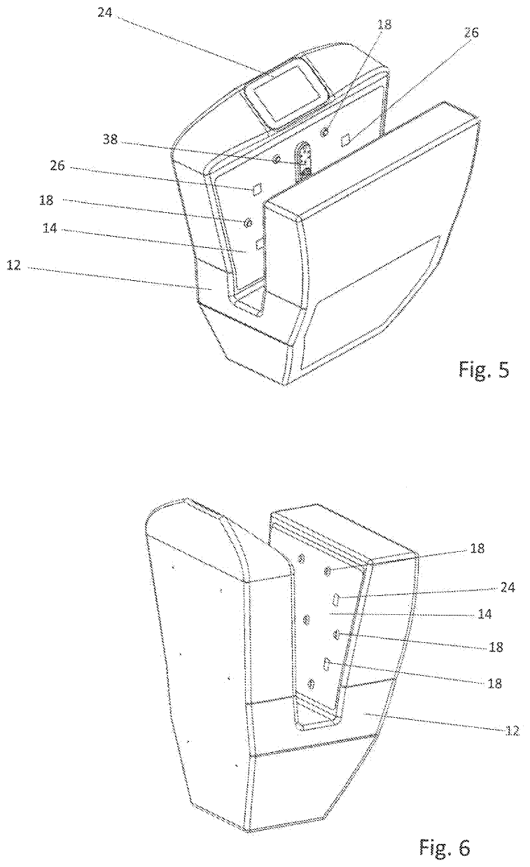

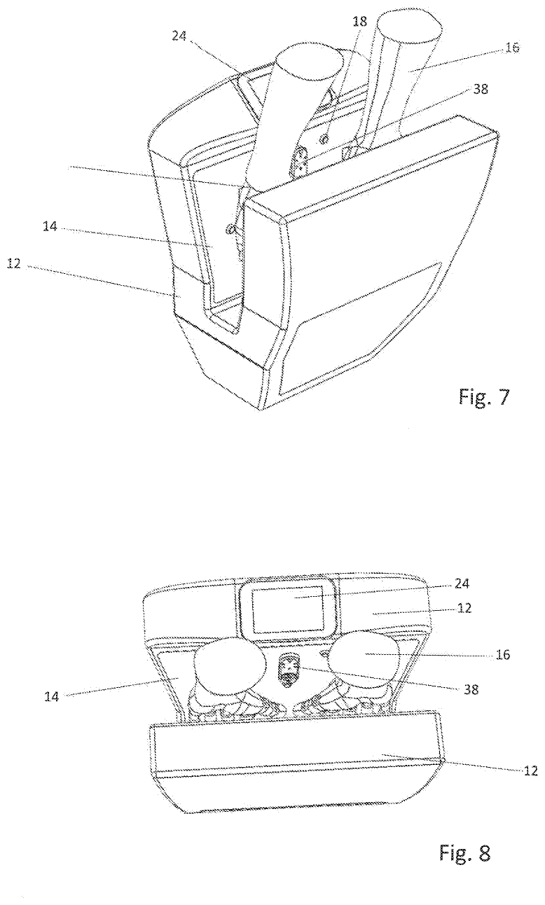

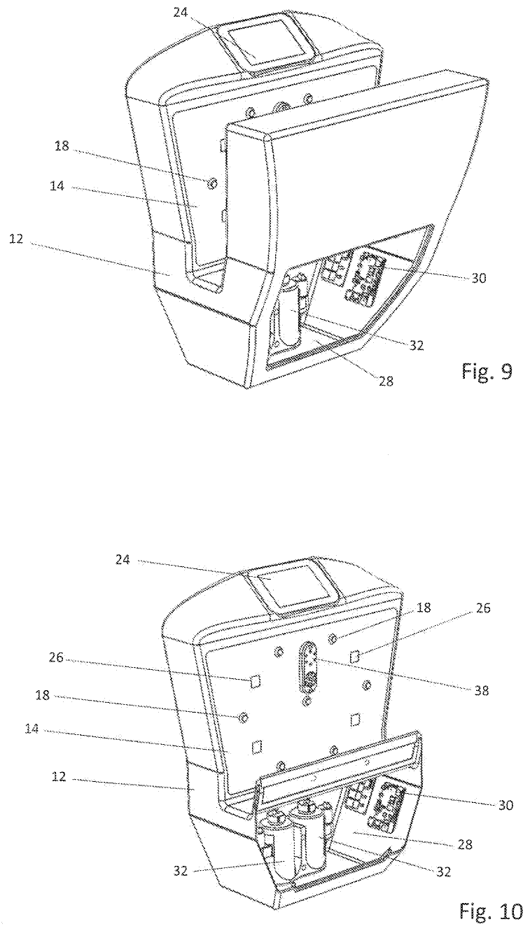

FIGS. 5 to 10 show another embodiment of the device configured for hands to be held in a vertical position in the hand sanitation and sensing area 14, which contains a plurality of cameras 26 (four on each wall of the area) and a plurality of dispensing nozzles 18. The device is shown with a sterilising light source 38, as mentioned above. A display device 24 is arranged on the top of the device, facing the user. FIG. 6 shows a different view, showing the cameras 26 and nozzles 18 on the fore wall of the sanitation and sensing area. FIG. 7 illustrates how hands 16 would be positioned in the device and FIG. 8 shows a top-view of the device, with inserted hands 16. In FIG. 9 the inside compartment/cavity 28 is shown opened (front opening panel removed) giving access to a sanitising agent canister 32 and electronic circuitry 30 (e.g. for replacement of the canister). FIG. 10 shows a view where the entire fore part of the device is not shown to give a better view of the aft wall of the sanitation and sensing area 14 and also exposing more of the compartment/cavity 28, where two sanitation agent canisters 32 are visible.

### Sensing Arrangement—Specific Detail

The sensing arrangement 26 may comprise one or more sensors located within the sanitisation and sensing area 14 such that the user's hands 16 can be sensed and data relating to the characteristics of interest obtained. In the embodiment shown, the sensing arrangement 26 comprises a visual system in which a plurality of cameras 26′ are configured to capture still or moving images of the user's hands 16 once inserted.

As can be seen there are in the illustrated embodiment four cameras 26′ on each of the upper and lower walls of the sanitisation and sensing area 14. The cameras 26′ may be configured to capture images of the palmer and dorsal surfaces of the hands 16 and also the sides of the hands 16 can be imaged. As such, areas on the sides of the fingers or thumbs or hands may be captured and analysed for contaminants or wounds.

In some embodiments, the cameras 26′ may comprise wide angled lens and may combine to provide multiple overlapping fields of view to enable a three-dimensional representation of the hands 16 to be constructed. Providing a three-dimensional image of the hands 16 may be useful for determining the position and posture of the hands 16 or for providing a map of the hands 16 including, for example, an overlay of any contaminants or foreign objects.

The cameras 26′ may be provided in suitable locations to allow the required image capture. In the example shown, there are two cameras 26′ placed below and two cameras 26′ placed above each hand in line with the longitudinal axis of the hand 16 but this is not a limitation and the number and position of the cameras 26′ may differ in other embodiments. The cameras may be arranged so that are moveable, for example, to accommodate position and/or size of inserted hands. Thus, in some embodiments one or more camera may be arranged on a moveable member, e.g. a member that can slide along rails such as vertical rails or a horizontal rail, e.g. a circular or elliptical rail. Thus, in some embodiments one or more camera can be moved during a sensing/detecting operation to encircle one or both hands. In some embodiments, cameras and nozzles are arranged on the same one or more moveable member.

The cameras 26′ may be configured to detect visible light. The visible light images may be used, for example, to show the position of the hands to a user on the user interface. In other embodiments the visible images may be used to determine the position and posture of the hands 16 using a suitable algorithm, such as a machine learning algorithm. Once the position of the hands 16 has been determined, audible and/or visual commands can be provided to a user to allow the hands 16 to be repositioned. For example, an image of the hands 16 may be displayed on the display device 24 in relation to a desired position represented by alignment marks or a ghost image of correctly positioned hands, for example.

In addition to using visible light, or as an alternative, the cameras 26′ may be configured to detect wavelengths other than visible light. Other wavelengths may be useful for detecting contaminants, for example. Wavelength specific imaging to detect contaminants is known and may include imaging for example in the wavelengths of between 10 nm and 400 nm (UV wavelengths). In some embodiments, the cameras 26′ may comprise filters so that the useful wavelengths might be separated out before being transmitted to a suitable image detector. In some embodiments, a plurality of wavelength specific cameras may be detected.

In order to improve imaging, the device 10 may include one or more light sources (not shown). The light sources may output light at one or more required wavelengths and may be LEDs. Such light sources are known in the art and will not be described further. As will appreciated, in some embodiments light sources are provided in the device that emit specific wavelengths that allow detection of certain impurities.

It will be appreciated that cameras 26′ may be exposed to the sanitising agent when being dispensed. As such the camera lenses may be configured to prevent an accumulation of sanitising agent. The camera lenses may, for example, comprise a hydrophobic coating and/or a heating device configured to evaporate any accumulated sanitising agent. In some embodiments, the cameras may comprise a mechanical shutter which is deployable during the dispensing of the sanitising agent or a mechanical wiper to clean the lens when required.

It will be appreciated that the sensing arrangement 26 may comprise one or more processors and/or controllers to enable the images to be captured and processed. In addition, the one or more processors or controllers may be configured to determine one or more of the characteristics of the hands for use in the sanitising process. The processors and controllers may form part of the cameras 26′ and/or be provided by a central controller or processor on the electronic devices 30 provided in the cavity 28. Additionally or alternatively, the processing of images and determination of the characteristics may be achieved with a remote computing device such as a server. These aspects are disclosed more fully below.

### Dispensing Device

The hand sanitising and hand hygiene monitoring device 10 comprises one or more dispensing device 18 for dispensing in a controlled manner the sanitising agent on to the hand such that the hand is sanitised. The dispensing device comprises at least one nozzle for dispensing and distributing the sanitiser. The specific way in which the sanitising agent is dispensed will be dependent on the type of sanitiser used and the dispersion which is desired and needed for complete sanitation. In some embodiments, the sanitising agent 14 may be provided as a spray or mist in which one more jets or a mist of sanitising agent is dispensed on the hands 16, in a suitably controlled manner as described above. The dispensing devices 18 may be distributed throughout the hand sanitisation and sensing area 14 and may include an atomiser nozzle or a directable nozzle such that the sanitising agent can be directionally dispensed. Thus, the direction of directable nozzles could be adjusted automatically based in obtained information e.g. size and/or position of hands, sensed contaminants, etc. Hence, where a user's hands 16 are not located correctly, the sanitising agent can be directed to the correct area for effective distribution, rather than applying an increased amount in the wrong area to offset the positional inaccuracy. In embodiments with directional nozzles, it may also be preferable to provide increased amounts of sanitising agent to areas which are likely to harbour more pathogens, such as around jewellery or wounds (but generally and preferably, as discussed above, the user will be prompted to remove any jewellery).

In some embodiments the device can hold more than type of sanitising agent 14 in separate canisters and the system can be configured to determine and select the most suitable type based on real-time data. For example, if the device detects a wound on a healthcare worker's hand, it might select a sanitizing fluid that contains a wound-friendly antiseptic, or the device may have learned personal preferences of the particular user

The dispensing device 18 shown in FIGS. 1 to 3 comprises atomising nozzles 18′ which eject a spray of mist toward the hands 16. There are two nozzles 18′ provided for each side of each hand by this is not a limitation and a different number nozzles may be provided and the location may vary. In some embodiments the device uses easily replaceable nozzle heads that can be e.g. magnetic or fastened with snap-on mechanism, with such configuration a clogged or defective nozzle can be readily replaced on-site by non-technical personnel.

It will be appreciated that the dispensing device 18 may comprise a pump, such as mentioned above, a source of sanitising agent such as a replaceable canister 32 or cartridge, a controller 30 configured to control the dispensing of the sanitising agent.

### User Interface

As noted above, a user interface may be utilised to provide information and/or instructions to a user. In the embodiment of FIGS. 1 to 4 there is provided a display device 24 in the form of a screen, such as an LCD or LED matrix, which allows still or moving images to be displayed to a user. In addition to this, or alternatively, the user interface may comprise one or more speakers (not shown) for issuing audible instructions or information.

In some embodiments, the hand sanitising and hand hygiene monitoring device 10 may be configured to identify the presence or specific identity of a user. Hence, the device 10 may comprise one or more cameras (nor shown) to capture an image of a user, for example, a user's face or part thereof such as one or both eyes or retinas. The user image may be used in facial recognition algorithm or retinal scanning recognition to determine the identification of the user. In some embodiments, the device 10 may comprise a reader (not shown) configured to read an electronic tag provided by a user. The electronic tag may be a key fob or mobile phone running a suitable app, for example. The electronic tag may be associated with the user to enable the user to be identified. The reader may comprise a short-range receiver to allow interaction using a short-range communication protocol such as Bluetooth low energy, BLE or a near field communication, NFC, for example.

In some embodiments, the user interface 22 may comprise one or more cameras or detectors, such as an near infra-red detector to determine when a user is in close proximity to the device, or when a user's hands have been inserted into the sanitisation and sensing area. It will be appreciated that one or more user detection cameras may be placed in any suitable location that allows them to obtain the required data, be it infra-red or visible light.

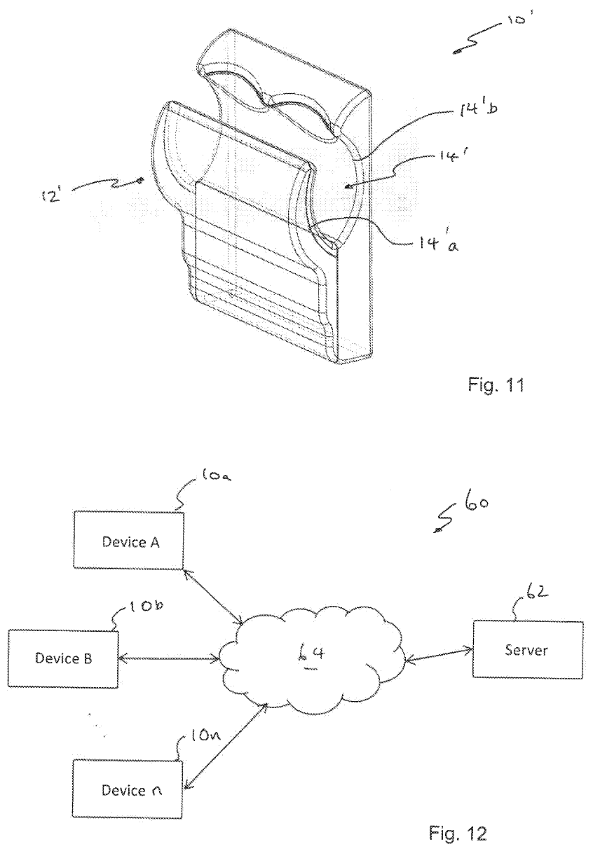

Turning to FIG. 11, there is shown an alternative hand sterilising device 10′ which comprises a sterilisation area 14′ between fore 14′a and aft 14′b walls. The sterilisation area 14′ includes an insertion aperture towards an upper end thereof such that a user inserts their hands from above and extend vertically into the sanitisation and sensing area 14′. The fore 14′a and aft 14′b walls of the sterilisation area 14′ curve away from the insertion aperture and each other prior to curving inwards to join at a bottom edge of the sterilisation area 14′. As such, the sterilisation area 14′ comprises a rounded profile in the vertical transverse (i.e. median) plane with a broadest dimension at approximate mid-height, and rectangular frontal section. The design of housing 12′ shown in FIG. 11 is slimmer that the embodiment of FIGS. 1 to 3 and so projects into the room where it is located less which may be advantageous.

The nozzles and sensors are not shown in the embodiment of FIG. 11, but it will be appreciated that they will be provided in suitable locations within the sterilisation area 14′ to enable the required imaging and dispensing of sanitising agent.

The user interface may comprise an audible output only in FIG. 11. Hence, upon detecting the position and posture of the hands, a user may be provided with audible instructions in relation to repositioning rather than visual. However, it will be appreciated that visual indicators may be provide on the upper surface. For example, the user may be presented with different coloured LEDs to indicate whether the position of the hands is suitable, or whether a foreign object has been detected and requires removal prior to sterilisation.

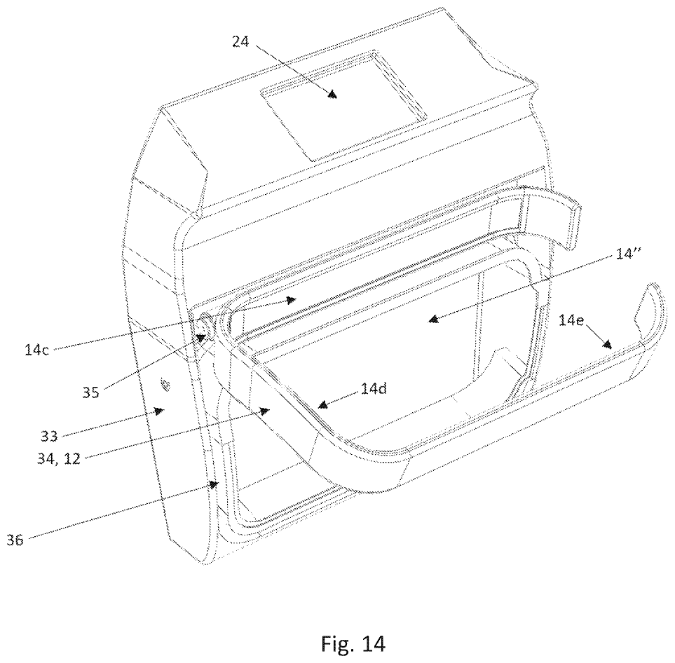

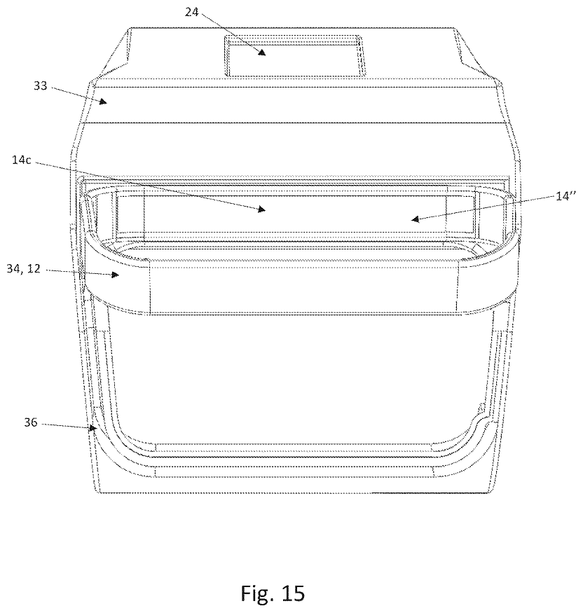

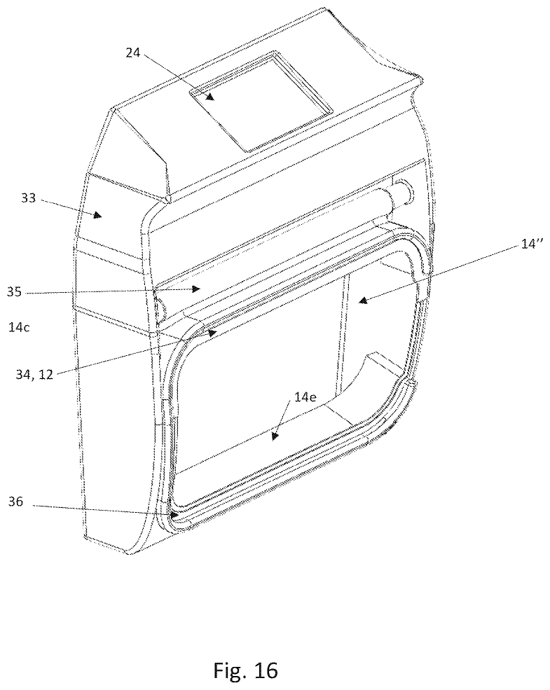

Another slim embodiment in shown in FIGS. 14-16. In this embodiment the device comprises a frame 34 that defines a perimeter (substantially horizontally) of the sensing and sterilisation area 14″. The frame thus can be seen as defining walls 14c, 14d, 14e that hold nozzles and camera sensors. The frame 34 is held by a wall unit 33 via a rotational axis 35 such that the frame can “fold” back into a recess 36 of the wall unit 33 when not being used; the frame is then in a slim position or closed position (shown in FIG. 16), and when the device is activated the frame folds out to an open position (FIGS. 14 and 15). The frame may have an opening on either side, to case insertion of the hands. This embodiment may require the user to move her hands through the frame (typically vertically downwardly) in order for the sensors and nozzles to fully image and sanitise the hands. In such case, the device will typically give the user instructions, e.g. informing her if hands are moved to quickly or not sufficiently long distance.

### System

FIG. 12 shows a schematic representation of a hand sanitising system 60 which may comprise one or more of the hand sanitising and hand hygiene monitoring devices 10a-n disclosed herein and a server 62, which are connected via a suitable communications network 64.

The system 60 may relate to a healthcare premises having multiple devices 10a-n or multiple premises having either single or multiple devices. In other embodiments the system may relate to pharmaceutical production premises, biochemical or biological research or production premises, food production or research premises or other premises or facilities where strict hygiene requirements are in place. The server 62 may be owned or controlled by a third party separate from the premises in which the devices 10a-n are installed. The server 62 may be located at the same premises as the devices or may be remote. The server 62 may serve several clients with processing and data handling capabilities required to fulfil the methods described herein. It will be appreciated that the arrows generally show a transmission of data from one entity to another.

The system 60 may be part of a health care management system (e.g. as part quality management system) which is configured to monitor and control hand sanitisation in one or more healthcare settings or other alternative management system configured for other alternative facilities such as mentioned above. Data acquired by one or more of the hand sanitising and hand hygiene monitoring devices 10a-n may be sent to the server 62. The data may relate to the operation of the device. For example, the data may be image data relating to the hands 16 such that the server 62 can analyse the images and determine whether the position is correct or requires adjustment, or whether the hands include foreign objects or contaminants, as previously described.

For information tracking purposes, the data transmitted to the server 62 may comprise identification information relating to the specific device, the location, the time of use or any other information which may be useful. In some embodiments, the data may include user data such that statistical information may be determined from the data of multiple devices, possibly from multiple different sites. In some embodiments, the data may include user identification data such as a number or other unique identifier provided by a user and/or one or more images of a user in order to allow facial recognition to carried out.

It will be appreciated that providing a central server allows the processing power required within the hand sanitisation devices to be reduced. This may be useful for saving cost and operating power for the individual hand sanitisation devices 10a-n.

In some embodiments, the data may relate to the sanitisation requirements for an individual user. As such, the devices 10a-n may acquire data from a user relating to characteristics of the user's hands 16, and the server 62 may determine from that data the sanitisation requirements, e.g. amounts of sanitising agent required and/or the dispensing locations. Once the hand sanitisation requirements have been determined, these may be transmitted to the hand sanitisation device 10a-n for implementing a hand sanitisation procedure.

The connection between the server 62 and the devices 10a-n may be hard-wired or wireless and achieved using conventional networking technology. Thus, the hand sanitising device 10a-n may communicate with the server 62 via the internet, a wide area network, a wireless local area network, or short-range wireless communications using known standards and/or protocols, such as TCP/IP, WiFi (RTM) or Bluetooth (RTM), for example.

The server 62 may be configured to provide information about a healthcare facility to an end user. For example, the system 60 may be configured to present information relating to the position of sanitisation devices 10a-n, the status of devices 10a-n, for example, sanitising agent levels (e.g. giving a warning message when the level in a particular device is below a minimum threshold level), maintenance requirements etc., use data including frequency of use, duration of use, time of use; user profiling including the presence of foreign objects and contaminants and user identification which may be used to assess particular user performance in terms of frequency of use, contaminants, foreign objects etc.

The server 62 may comprise a suitable output such as a display device to present the information to an end user, or the information may be provided to another computing device on the network (not shown) which can be used to present the information to the end user.

It will be appreciated that the server 62 may be conventional and include one or more processors configured to carry out any of the actions disclosed herein and will include a memory which may store instructions which, when executed by the processor, cause the performance of any of the method steps disclosed herein.

The system with the described server can advantageously be used with machine learning algorithms as further discussed below.

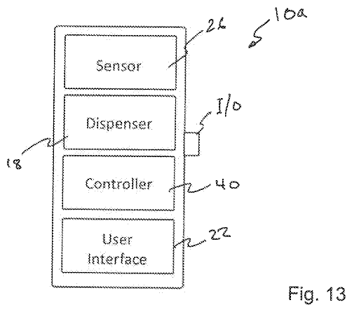

FIG. 13 shows a schematic representation of a hand sanitising and hand hygiene monitoring device 10a comprising the sensor arrangement 26 and dispensing device 18, as previously described, an optional conventional input/output module I/O for transmitting and receiving data to and from the server 62, a user interface 22 as previously described; and, a controller 40.

The controller 40 may comprise one or more processors and a data store which may store instructions which, when executed by the processor cause the performance of any of the method steps disclosed herein. The data store may be a memory which may comprise: volatile memory, for example, one or more dynamic random access (DRAM) modules and/or static random access memory (SRAM) modules; and/or non-volatile memory, for example, one or more read only memory (ROM) modules, which for example may comprise a Flash memory and/or other electrically erasable programmable read-only memory (EEPROM) device. The controller can be part of a server, as escribed above, or the functions of the controller can be divided between a local controller of the/each device and central controller of a server. It follows that some or all of the mentioned functions can be performed by a controller on a server.

The controller 40 may comprise a conventional processor which may include one or more microprocessors executed in hardware or software, for example. The processor may include at least one microprocessor and may comprise a single core processor, may comprise multiple processor cores (such as a dual core processor or a quad core processor), or may comprise a plurality of processors (at least one of which may comprise multiple processor cores). In various examples, the controller may comprise: control circuitry; and/or processor circuitry; and/or at least one application specific integrated circuit (ASIC); and/or at least one field programmable gate array (FPGA); and/or single or multi-processor architectures; and/or sequential/parallel architectures; and/or at least one programmable logic controllers (PLCs); and/or at least one microprocessor; and/or at least one microcontroller; and/or a central processing unit (CPU); and/or a graphics processing unit (GPU), and/or transceiver(s) to perform the methods.

### ML Algorithm

In some embodiments, the memory may store a machine learning algorithm. The machine learning algorithm may be used to determine the position and posture of the hands, the detection of the foreign matter or contaminants, and/or the dispensing requirements for the sanitiser. The machine learning algorithms may comprise well known algorithms for facial recognition, the detection of foreign matter or contaminant and/or the dispensing of fluid. Thus, in some embodiments a supervised image classification algorithm is used (in such algorithm a large amount of data is collected and labelled. This may include using Convolutional Neural Networks CNN). In other embodiments algorithms such as YOLOR (v3,v4) or Mask-R-CNN are used. Inference timing may be based on the MS COCO benchmark (which is different depending on if CPU or GPU is used). Extensive training data has been and will continue to by collected by the present inventors. The machine learning algorithm can in some embodiments be used to improve the instructions provided to the users as it can learn to optimise the sanitation process, e.g. dispensing time, speed at which hands may need to be moved, position of hands, etc. The system and device can in some embodiments apply haptic feedback, such that the device provides tactile signals to the user's hands.

The present disclosure provides a hand sanitising and hand hygiene monitoring device which is configured to provide improved hand sanitisation for users. The hand sanitising and hand hygiene monitoring device is primarily envisaged for use in healthcare settings but this is not a limitation and the hand sanitising and hand hygiene monitoring device may be used elsewhere, such as in facilities mentioned above.

The device and/or system of which the device is part of can in some embodiments, in particular where machine learning techniques are applied, analyse patterns in a facility's operation, and thus, for example, predict the most effective times for hand sanitization. This could involve identifying peak times of patient contact or procedures that may require enhanced sanitization beforehand, observe at what times or in what situations or locations enhance sanitation is critical and/or risk of compromised sanitation elevated.

In these embodiments, the AI algorithms which are incorporated and part of the provided QMS are trained on machine learning models specializing in image and pattern recognition. This allows the system to better recognize unwanted items like nail polish, rings, and visible wounds through real-time scanning. The AI is constantly learning, refining its recognition capabilities based on the continuous influx of data.

For sanitizing fluid application, the AI-enhanced QMS utilizes sensor data and previously learned sanitization patterns. The system calculates the hand's surface area and shape, determining the optimal distribution of sanitizing fluid, ensuring uniform and efficient application, and improving the overall effectiveness of the sanitization process.

Advantageously, the AI within the QMS preferably also has the ability to analyse long-term data trends, predict common hygiene discrepancies, and suggest pre-emptive measures. This innovative approach contributes to maintaining optimal hand hygiene among healthcare workers, and serves as an effective solution to mitigate healthcare-associated infections.

The one or more embodiments are described above by way of example only. This patent application encompasses a comprehensive description of the present invention and some of its possible applications. However, it is critical to acknowledge that these embodiments are some of many possible implementations of the invention. Any future alterations or modifications to the AI algorithms, IoT devices, cloud-based QMS, or sanitization techniques envisioned by those skilled in the art fall within the purview of the invention and are covered by the appended claims that define the invention in its broadest sense. Many variations are possible without departing from the scope of protection afforded by the appended claims.

1-35. (canceled)

36. A hand sanitising and hand hygiene monitoring device (10), for automatically sanitising hands of a user, comprising:
a housing (12) comprising a hand sanitisation and sensing area (14) configured so that a user can position his hands in the hand sanitisation and sensing area for sanitising and sensing the hands;
wherein the housing comprises a moveable frame joined to a wall unit of the device with a fastening arrangement such that the frame can fold out from a closed position to an open active position where the user can put his hands through the frame, the frame defining walls (14c, 14d, 14e) that define the sanitisation and sensing area;
a sensing arrangement configured to obtain data relating to a characteristic of the user's hand located within the sanitisation and sensing area, the sensing arrangement comprising one or more sensors located on the walls of the frame; and,
a dispensing device (18) for dispensing and distributing in a controlled manner a sanitising agent in the sanitising and sensing area, facilitating full surface coverage such that an inserted hand of the user can be sanitised, the dispensing device comprising at least two nozzles (18) for dispensing hand sanitising agent,
the nozzles configured such that one of said nozzles is directed to the palmer side of an inserted hand and the other of said oppositely arranged nozzles is directed to the dorsal side of said inserted hand;
a user interface configured to provide information relating to the characteristic to the user and optionally statistical information or other user-specific information, wherein the user interface is configured to provide positional information to the user and, optionally, directions for a user to reposition the user's hand and/or provide instructions to a user to remove one or more foreign objects,
the hand sanitising and hand hygiene monitoring device configured to determine whether the application of the hand sanitising agent is satisfactory by analysing the position of the hands relative to the dispensing nozzles.

37. The hand sanitising and hand hygiene monitoring device according to claim 36, wherein the characteristic relates to one or more from the group comprising: a position of the user's hand relative to the dispensing device; a size of the user's hand; a contaminant on the user's hand; a wound on the user's hand; and a foreign object on the user's hand.

38. The hand sanitising and hand hygiene monitoring device according to claim 36, wherein the user interface is configured to provide instructions to a user to remove one or more foreign objects.

39. The hand sanitising and hand hygiene monitoring device according to claim 36, wherein the user interface is configured to provide information or instructions in relation to sensed contaminants or foreign objects.

40. The hand sanitising and hand hygiene monitoring device according to claim 36, wherein the user interface comprises either or both of an audible or visual output device.

41. The hand sanitising and hand hygiene monitoring device according to claim 40, wherein the user interface comprises a display device provided on the housing.

42. The hand sanitising and hand hygiene monitoring device according to claim 36, wherein the housing comprises an opening into which the user can insert their hand, the opening being located on a front or upper surface of the housing.

43. The hand sanitising and hand hygiene monitoring device according to claim 36, wherein the device is configured so that it can move the frame during a sanitation cycle to aid full surface coverage of hands.

44. The hand sanitising and hand hygiene monitoring device according to claim 36, wherein the main wall unit comprises a recess or compartment that receives and situates the frame when in a closed position, the device comprising a rotational axis and/or hinges that hold the frame such that the frame can be folded out from the closed position to the open position.

45. The hand sanitising and hand hygiene monitoring device according to claim 36, wherein the sensing arrangement comprises a plurality of cameras configured to capture images of the user's hand when in the sanitisation and sensing area.

46. The hand sanitising and hand hygiene monitoring device according to claim 45, wherein the plurality of cameras are configured to obtain a plurality of different images from different locations and optionally different time points.

47. The hand sanitising and hand hygiene monitoring device according to claim 36, further comprising at least one processor configured to determine the characteristic from the obtained data, and/or to process transmitted or received data.

48. The hand sanitising and hand hygiene monitoring device according to claim 36, further comprising a transmitter or wired connection configured to transmit data relating to the sensed characteristic to a server.

49. The hand sanitising and hand hygiene monitoring device according to claim 47, wherein the at least one processor or server is configured to construct an image of the user's hand including information on the characteristic and the sensing arrangement comprises a plurality of cameras configured to capture images of the user's hand when in the sanitisation and sensing area.

50. The hand sanitising and hand hygiene monitoring device according to claim 49, wherein the image of the user's comprises indicia relating to the position or distribution of foreign objects, contaminants or a wound.

51. The hand sanitising and hand hygiene monitoring device according to claim 45, wherein the cameras are configured to image the palmer side and dorsal side of the user's hand.

52. The hand sanitising and hand hygiene monitoring device according to claim 45, wherein the plurality of cameras comprise at least one camera configured to detect light having a wavelength of between 10 nm and 400 nm.

53. The hand sanitising and hand hygiene monitoring device according to claim 45, wherein the cameras comprise one or more lens or lens cover through which an image is captured, wherein said lens or lens cover is configured to prevent the accumulation of hand sanitising agent thereon.

54. The hand sanitising and hand hygiene monitoring device according to claim 36, further comprising a UV light source for sterilising the sanitisation and sensing area.

55. The hand sanitising and hand hygiene monitoring device according to claim 36, wherein the nozzle comprises a spray head for ejecting a spray of sanitising agent.

56. The hand sanitising and hand hygiene monitoring device according to claim 55, further comprising a controller for controlling the amount and/or a spray pattern of the sanitising agent, wherein the amount and/or spray pattern is determined using the obtained characteristic.

57. The hand sanitising and hand hygiene monitoring device according to claim 48, wherein the server is configured to receive the obtained data and determine the characteristic and/or provide statistical information relating to the obtained data.

58. The hand sanitising and hand hygiene monitoring device according to claim 57, wherein the server is configured to determine the characteristic using a machine learning algorithm.

59. The hand sanitising system comprising the hand sanitising and hand hygiene monitoring device according to claim 36 and a server, wherein the server is configured to provide information in relation to the obtained data and/or characteristics of one or more users.

60. The hand sanitising system according to claim 59, comprising a plurality of hand sanitising and hand hygiene monitoring devices, wherein said server comprises software for collecting and providing information in relation to the obtained data and/or characteristics, including statistical information for hygiene management and monitoring.

61. The method of sanitising hands with a hand sanitising and hand hygiene monitoring device as defined in claim 36, the method comprising
a user inserting at least one hand in a sanitisation and sensing area of the device,
obtaining data with sensors wherein the data relates to a characteristic of the at least one hand,
dispensing and distributing in a controlled manner a sanitising agent in the sanitising and sensing area onto the hand, and,
such that the hand is fully sanitised.

62. The method according to claim 61, wherein the characteristic relates to one or more from the group comprising: a position of the user's hand relative to the dispensing device; a size of the user's hand; a contaminant on the user's hand; a wound on the user's hand; and a foreign object on the user's hand.

63. The method according to claim 61, comprising providing instructions to the user selected from one or more of:
directions for the user to reposition his or her hand,
instructions to the user to remove one or more foreign objects,
instructions to the user that a wash of hands is necessary before sanitation can be completed.

64. The method according to claim 63, wherein said instructions are provided via a user interface selected from one or both of an audible or visual output device.

65. The method according to claim 61, comprising activating the device, which triggers the device to make available the sanitisation and sensing area by rotatably folding out a frame defining the sanitisation and sensing area.

66. The method according to claim 61, further comprising disinfecting the sanitisation and sensing area after a user has sanitised his hands.
